(19) United States
(12) Patent Application Publication
(10) Pub. No.: US 2009/0163577 A1
(43) Pub. Date: Jun. 25, 2009

(54) METHODS AND COMPOSITIONS FOR ANTAGONIZING ANTI-APOPTOTIC Bcl-2-FAMILY PROTEINS

(75) Inventors: John C. Reed, La Jolla, CA (US); Dayong Zhai, La Jolla, CA (US)

Correspondence Address: BioTechnology Law Group;12707 High Bluff Drive, Suite 200, San Diego, CA, 92130-2037, US

(73) Assignee: Burnham Institute for Medical Research, La Jolla, CA (US)

(21) Appl. No.: 12/325,315

(22) Filed: Dec. 1, 2008

Related U.S. Application Data
(60) Provisional application No. 61/005,303, filed on Dec. 3, 2007.

(51) Int. Cl. A61K 31/35; C12Q 1/18; G01N 33/574; C07D 493/02; A61P 35/00

(52) U.S. Cl. 514/453; 435/32; 436/501; 549/382

(57) ABSTRACT

The cytotoxic natural product gambogic acid (GA) competes for BH3 peptide binding sites on several anti-apoptotic members of the Bcl-2 family of proteins and neutralizes the ability of these proteins to suppress release of apoptogenic proteins from isolated mitochondria. Structure-function analysis of GA using analogs suggested a general correlation between BH3 competition and cytoxicity activity. Compositions and methods are provided for using GA and its derivatives for treating cancer and for discovering other compounds that are useful for treating cancer through their interaction with Bcl-2-family proteins.

### RELATED APPLICATIONS

The present application claims priority to the U.S. Provisional Application Ser. No. 61/005,303, filed Dec. 3, 2007 by Reed et al., and entitled “METHODS AND COMPOSITIONS FOR ANTAGONIZING ANTI-APOPTOTIC Bcl-2-FAMILY PROTEINS,” which in incorporated by reference herein in its entirety, including the drawings.

### STATEMENT OF GOVERNMENT RIGHTS

The invention was supported, at least in part, by a grant from the Government of the United States of America (grants no. CA-113318 and CA-55164 from the National Institutes of Health). The Government has certain rights to the invention.

### TECHNICAL FIELD

The present invention relates to cancer therapy, particularly to compounds that have cytotoxic activity against cancer cells.

### BACKGROUND

Gambogic acid (GA) is a medicinal compound derived from the gamboges resin of the tree, Garcinia hanburyi. GA has documented cytotoxic activity against tumor cell lines in culture, with concentrations required for killing 50% of cells (Lethal Dose 50% [LD₅₀] of ˜1 μM (Zhang et al., Bioor. Med. Chem. 12:309-317, 2004; Zhao et al., Biol. Pharm. Bull. 27:998-1003, 2004). In contrast, GA is reportedly well tolerated in rats (Zhao et al., Biol. Pharm. Bull. 27:998-1003, 2004), suggesting that a therapeutic window might be identified at which tumor but not normal cells are killed.

The mechanism by which GA kills tumor cells lines involves apoptosis, a cell death processing involving caspase-family proteases. In fact, GA was identified as an active compound in a cell-based high-throughput screening (HTS) assay that measured caspase activation (Zhang et al., Bioor. Med. Chem. 12:309-317, 2004). Among the regulators of apoptosis are Bcl-2-family proteins. Humans have six genes encoding distinct anti-apoptotic Bcl-2-family proteins: Bcl-2, Bcl-X_{L}, Mcl-1, Bfl-1, Bcl-W, and Bcl-B (Cory and Adams, Nat. Rev. Cancer 2:647-656, 2002; Reed, Cancer Cell 3:17-22, 2003). These proteins typically localize to intracellular membranes, especially mitochondrial membranes, where they have been shown to block the release of apoptogenic proteins such as cytochrome c, SMAC Endonuclease G, and AIF (Du et al., Cell 102:33-42, 2000; Kroemer and Reed, Nat. Med. 6:513-519, 2000; Verhagen et al., Cell 102:43-53, 2000). Several anti-apoptotic Bcl-2-family proteins are known to become pathologically over-expressed in human cancers, conferring apoptosis-resistant phenotypes (Raffo et al., Cancer Res. 55:4438-4445, 1995; Furuya et al., Clin. Cancer Res. 2:389-398, 1996; Lomo et al., Cancer Res. 56:40-43, 1996; Schimmer et al., Curr. Treat. Options Oncol. 4:211-218, 2003; Letai et al., Cancer Cell 6:241-249, 2004; Andersen et al., Blood 105:728-734, 2005).

The anti-apoptotic proteins are neutralized endogenously by proteins containing an α-helical interaction motif, known as BH3 (Cory and Adams, Nat. Rev. Cancer 2:647-656, 2002; Reed, Nature 387:773-776, 1997; Adams and Cory, Science 281:1322-1326, 1998; Reed, Am. J. Pathol. 157:1415-1430, 2000). Synthetic BH3 peptides bind anti-apoptotic Bcl-2-family proteins with nanomolar affinities, promoting apoptosis (Kuwana et al., Mol. Cell. 17:525-535, 2005; Chen et al., Mol. Cell. 17:393-403, 2005). Non-peptidyl compounds have been identified that compete with BH3 peptides for binding to anti-apoptotic Bcl-2-family proteins, mimicking BH3 peptides and creating interest in development of these molecules as potential cancer therapeutics (Reed and Pellecchia, Blood 106:408-418, 2005; Zhai et al., “Comparison of chemical inhibitors of anti-apoptotic Bcl-2-family proteins,” Cell Death Differ. 13:1419-1421, 2006).

### SUMMARY OF THE INVENTION

We have discovered that gambogic acid (GA) competes for BH3 peptide binding sites on several anti-apoptotic members of the Bcl-2 family of proteins and neutralizes the ability of these proteins to suppress release of apoptogenic proteins from isolated mitochondria. There is a general correlation between BH3 competition and cytoxicity activity.

Accordingly, the present invention provides compositions and methods for using GA and its derivatives for treating cancer and for discovering other compounds that are useful for treating cancer through their interaction with Bcl-2-family proteins.

According to a first embodiment of the invention, methods are provided for identifying a test compound that is effective for treating a disease or condition of a mammal selected from the group consisting of a cancer, an non-cancerous proliferative condition, and an autoimmune disease, or disorder, the method comprising determine whether the test compound competes with gambogic acid or a gambogic acid derivative for binding to a Bcl-2-family protein. Such methods may comprise: (a) providing a first mixture comprising the Bcl-2-family protein and the test compound; (b) incubating the first mixture under conditions suitable for binding of the test compound to the Bcl-2-family protein; (c) adding the gambogic acid or the gambogic acid derivative to the mixture to produce a second mixture comprising the Bcl-2-family protein, the gambogic acid or the gambogic acid derivative, and the test compound; (d) incubating the second mixture under conditions suitable for binding of the gambogic acid or the gambogic acid derivative to the Bcl-2-family protein; and (e) measuring binding of the gambogic acid or the gambogic acid derivative to the Bcl-2-family protein in the second mixture. Alternatively, such methods may comprise: (a) providing a first mixture comprising the Bcl-2-family protein and the gambogic acid or the gambogic acid derivative; (b) incubating the first mixture under conditions suitable for binding of the gambogic acid or the gambogic acid derivative to the Bcl-2-family protein; (c) adding the test compound to the first mixture to produce a second mixture comprising the Bcl-2-family protein, the gambogic acid or the gambogic acid derivative, and the test compound; (d) incubating the second mixture under conditions suitable for binding of the test compound to the Bcl-2-family protein; and (e) measuring binding of the gambogic acid or the gambogic acid derivative to the Bcl-2-family protein.

In such methods, the gambogic acid or the gambogic acid derivative may comprise a detectable label such as, for example, a fluorochrome, biotin and a radiolabel. Where the detectable label is a fluorochrome, such methods may comprise determining whether the test compound competes with the gambogic acid or the gambogic acid derivative for binding to the Bcl-2-family protein by performing a fluorescence polarization assay, a time-resolved fluorescence resonance energy transfer assay, an AlphaScreen™ assay, or a scintillation proximity assay, for example, to measure binding of the gambogic acid or the gambogic acid derivative to the Bcl-2-family protein.

Such methods may further comprise contacting a cell of the mammal that expresses a Bcl-2-family protein with the test compound and determining whether the test compound is cytotoxic against the cell. Non-limiting examples of such cells are, for example, HeLa cells, HL60 cells, Jurkat cells, and PPC1 cells. Such methods may also comprise providing the cell in a well of a multi-well plate, and such methods are preferably automated.

In such methods, the mammal may be, for example, a human. The foregoing methods are useful for identifying a test compound that is effective for treating: a cancer including, but not limited to, pancreatic cancer; bladder cancer; colorectal cancer; breast cancer, including metastatic breast cancer; prostate cancer; renal cancer; hepatocellular cancer; lung cancer; ovarian cancer; cervical cancer; gastric cancer; esophageal cancer; head and neck cancer; melanoma; neuroendocrine cancer; brain tumors; bone cancer; soft tissue sarcoma; acute myeloid leukemia; chronic myelogenous leukemia; acute lymphoblastic leukemia; chronic lymphocytic leukemia; Hodgkin's disease; non-Hodgkin's lymphoma; B-cell lymphoma; T-cell lymphoma; multiple myeloma; Waldenstrom's macroglobulinemia; myelodysplastic syndromes; and myeloproliferative syndromes; a non-cancerous proliferative condition including but not limited to a skin disease, a keloid, a scar and benign prostatic hypertrophy; or an autoimmune disorder including but not limited to rheumatoid arthritis, lupus, multiple sclerosis, Sjögren's syndrome, spondylosing ankylitis, psoriasis, graft rejection and graft versus host disease.

According to another embodiment of the invention, methods are provided for identifying a compound that is effective for treating a mammal having a disease or condition selected from the group consisting of a cancer, a non-cancerous proliferative condition, and an autoimmune disorder comprising comparing a three-dimensional structure of said compound when bound to a Bcl-2-family protein to a three-dimensional structure of gambogic acid bound to the Bcl-2-family protein.

According to another embodiment of the invention, compositions are provided comprising an amount of gambogic acid or a gambogic acid derivative that is effective in treating a mammal (such as, for example, a human) having a disease or condition selected from the group consisting of a cancer, a non-cancerous proliferative condition, and an autoimmune disorder, such as those listed above. Such compositions may also comprise a pharmaceutically acceptable carrier. Such compositions may also comprise an amount of an additional active ingredient that is effective in treating the mammal having said disease or condition.

According to another embodiment of the invention, methods are provided for treating a cancer (such as those listed above) in a mammal in need thereof comprising administering to the mammal a composition comprising an amount of gambogic or a gambogic acid derivative that is cytotoxic against the cancer.

According to another embodiment of the invention, methods are provided for making a medicament useful in treating a cancer in a mammal in need of such treatment, the method comprising incorporating a compound that competes with gambogic acid or a gambogic acid derivative for binding to a Bcl-2-family protein in a cell of the cancer into a pharmaceutical composition comprising the compound and a pharmaceutically acceptable carrier.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE FIGURES

FIG. 1 shows that gambogic acid (GA) inhibits binding of FITC-BH3 peptide to anti-apoptotic Bcl-2 family proteins. (A) Structure of gambogic acid. (B-E) For peptide competition experiments in FPA mode, 100 nM of Bcl-2 family proteins, including Bcl-X_{L}, Bcl-2, Bcl-W, Bcl-B, Bfl-1 or Mcl-1, were incubated with various concentrations of gambogic acid (GA) for 2 min in PBS buffer, then 5 nM FITC-conjugated-Bid BH3 peptide was added. Fluorescence polarization (milli-Polars [mP]) was measured after 10 min. Interaction of gambogic acid with FITC-conjugated Bid BH3 peptide alone was used as a control. Note that background FP for these assays from FITC-BH3 peptide in the absence of Bcl-2 family protein is ˜50-70 mP (FPₘᵢₙ), while the maximum FP ranges from ˜130 to ˜190 mP (FPₘₐₓ), depending on the Bcl-2-family member.

FIG. 2 shows that a TR-FRET assay confirms that gambogic acid competes with BH3 peptide for binding to Bcl-X_{L}. (A) Binding of GST-Bcl-X_{L} to FITC-BH3 peptide was assayed by TR-FRET. The final reaction mixtures contain 10 nM of FITC-Bad BH3 peptide, 2 nM of anti-GST-Turbium, and various concentrations of GST-Bcl-X_{L} proteins in PBS buffer containing 0.005% Tween 20. (B) Analysis of GA competition with BH3 peptide for Bcl-X_{L} binding by TR-FRET. Reaction mixtures of final 20 μl volume contain 2 μl of various concentrations of GA with 10 nM FITC-Bad BH3 peptide, 2 nM anti-GST-Terbium, and 10 nM GST-Bcl-X_{L} protein in PBS buffer containing 0.005% Tween 20. The mixtures are incubated for 30 min at room temperature. TR-FRET signals were measured using excitation at 330 nm, emission for FITC signal at 490 nm, and emission for terbium signal at 520 nm. Data are presented as 520 nm/490 nm ratio (mean±std dev; n=3).

FIG. 3 shows that gambogic acid induces apoptosis of cancer cells. Gambogic acid was used to treat HL-60 (black symbols) or Jurkat (white symbols) cells at concentrations of 0, 0.1, 0.2, 0.5, 2, 5 μM (from left to right). Cells were collected after 20 hrs, stained with FITC-Annexin V and PI, and the percentage of Annexin V-positive/PT-negative cells was determined.

FIG. 4 shows the effects of Bcl-2 over-expression on Gambogic acid-induced cytotoxicity. Neo-control or Bcl-2 over-expressing HL-60 cells were treated with (A) gambogic acid or (B) Staurosporine at various concentrations, as indicated. After 8 hrs, the cells were collected, stained with FITC-Annexin V and PI, and the percentage of live cells was determined (Annexin-negative/PI-negative). Data are representative of at least 3 independent experiments.

FIG. 5 shows a comparison of cytotoxic activity and BH3 peptide displacement activity of Gambogic acid and analogs. (A) Structures of GA and GA analogs are depicted, and IC₅₀ values from FPAs are presented for competitive binding assays performed using Bfl-1 and FITC-Bid BH3 peptide. (B) Jurkat cells were treated with GA and analogs at concentrations of 0, 1, 5, 10, 20, 50 μM. Cells were collected after 20 hrs, stained with FITC-Annexin V and PI, and the percentages of live cells (Annexin-negative/PI-negative) were determined. Data are representative of at least three experiments.

FIG. 6 shows that gambogic acid kills mouse embryonic fibroblasts (MEFs) cells. MEF cells with wild-type (WT) (black symbols) or bax^{-/-}/bak^{-/-} (DKO) (white symbols) genotypes were treated with GA (A) or Staurosporine (B) at various concentrations. After 20 hrs, the cells were collected, stained with FITC-Annexin V and PI, and the percentage of Annexin V-negative/PT-negative (live) cells was determined. Data are representative of at least three experiments.

### DETAILED DESCRIPTION OF THE INVENTION

### Definitions

As used herein, “gambogic acid” refers to gambogic acid, and any pharmaceutically acceptable analogs, derivatives, salts, solvates and prodrugs thereof. Gambogic acid analogs and derivatives include, but are not limited, to those disclosed herein.

There are many functional groups in the structure of gambogic acid that can be modified. These include, but are not limited to, the carboxyl group, which can be converted to an ester, amide, ketone or alcohol and other functional groups. The ester and amide can also contain other functional groups, such as a carboxyl in an amino acid, for further modification; the hydroxy group, which can be converted to an ether or ester and other functional groups; the carbon-carbon double bond in the α,β-unsaturated ketone, which can react with a nucleophile, or be reduced to a carbon-carbon single bond, or be converted to an epoxide, and undergo other reactions; the carbon-carbon double bond in the α,β-unsaturated carboxyl, which also can react with a nucleophile, or be reduced to a carbon-carbon single bond, or be converted to a cyclopropane ring, and undergo other reactions; the two isoprene carbon-carbon double bonds, which can be reduced to a carbon-carbon single bond, or be converted to an epoxide, which can then undergo other reactions, or be cleaved to form an aldehyde or carboxyl group, which also can be modified to other functional groups; the carbon-carbon double bond in the left ring also can be reduced to a carbon-carbon single bond, or be converted to an epoxide, and undergo other reactions; the ketone group in the right ring can be reduced to an alcohol, or be converted to an oxime or a semicarbazone, or be converted to an amino group; the other ketone group also can be reduced, or be converted to other functional groups. Therefore, many derivatives of gambogic acid can be prepared.

In addition, analogs of gambogic acid, including isomorellin, morellic acid, desoxymorellin, gambogin, morelline dimethyl acetal, isomoreollin B, Moreollinc acid, gambogenic acid, gambogenin, isogambogenin, desoxygambogenin, gambogenin dimethyl acetal, gambogellic acid, hanburin (Asano et al., Phytochemistry 41:815-820 (1996)), isogambogic acid, isomorellinol (Lin et al., Magn. Reson. Chem. 31:340-347, 1993) and neo-gambogic acid (Lu et al., Yao Hsuch Hsuch Pao 19:636-639, 1984) can be isolated from gamboge. Other analogs of gambogic acid, including morellin, desoxymorellin, dihydroisomorellin (Bhat et al., Indian J. Chem. 2:405-409, 1964) and moreollin (Rao et al. Proc. Indian Acad. Sci. 87A:75-86, 1978), can be isolated from the seed of Garcinia morella. Morellinol can be isolated from the bark of Garcinia morella (Adawadkar et al. Indian J. Chem. 14B:19-21, 1976). Gaudichaudiones (A-H) and gaudichaudiic acids (A-E) can be isolated from the leaves of Garcinia gaudichaudii (Guttiferae) (Cao et al., Tetrahedron 54:10915-10924, 1998; Cao et al., Tetrahedron Lett. 39(20):3353-3356, 1998), and Wu et al., Planta Med. 68:198-203, 2002). Forbesione can be isolated from Garcinia forbesii (Leong et al., J. Chem. Res., Synop. 392-393, 1996). Bractatin, isobractatin, 1-0-methylbractatin, 1-0-methylisobractatin, 1-0-methyl-8-methoxy-8,8a-dihydrobractatin, and 1-0-methylneobractatin can be isolated from a leaf extract of G. bracteata (Thoison et al., J. Nat. Prod. 63:441-446, 2000). Novel gaudichaudiic acids (F-I) can be isolated from the bark of Indonesian Garcinia gaudichaudil (Xu et al., Organic Lett. 2:3945-3948, 2000). Scortechinones (A-C) can be isolated from twigs of Garcinia scortechinii (Rukachaisirikul et al., Tetrahedron 56:8539-8543, 2000). Gaudispirolactone can be isolated from the bark of Garcinia gaudichaudii (Wu et al., Tetrahedron Lett. 42:727-729, 2001). These gambogic acid analogs also can be used for the preparation of derivatives similar to gambogic acid. U.S. Patent Applications 20030078292, 20040082066, and 20070093456, for example, describe additional gambogic acid derivatives that may be employed in the method of invention including, without limitation: 2-(Morpholin-4-yl)-ethyl gambogate; 2-Dimethylaminoethyl gambogate; N-[3-(4-Methyl-piperazin-1-yl)-propyl]gambogamide; N-(3-Morpholin-4-yl-propyl)gambogamide; Methyl 37,38-Dihydroxy-gambogate; Methyl 37,38-Dihydroxy-9,10-dihydro-10-morpholinyl-gambogate; Methyl 20-Ethylaldehyde-9,10-dihydro-10-morpholinyl-morellinate; N-(4-Azido-2,3,5,6-tetrafluorobenzyl)gambogamide; N-(1,2-Dicarboxylethyl) gambogamide; and N-(4-Azidobenzohydrazide)gambogamide, among others.

The positions in gambogic acid are numbered according to Asano et al., Phytochemistry 41:815-820, 1996), and Lin et al., Magn. Reson. Chem. 31:340-347, 1993.

As used herein, “agent” refers to any substance that has a desired biological activity. An “anti-cancer agent” has detectable biological activity in treating cancer, e.g., in killing a cancer cell, treating or preventing cancer, reducing or stopping growth of a cancer, or reducing a symptom of a cancer, in a host.

As used herein, “effective amount” refers to an amount of a composition that causes a detectable difference in an observable biological effect, for example, a statistically significant difference in such an effect. The detectable difference may result from a single substance in the composition, from a combination of substances in the composition, or from the combined effects of administration of more than one composition. For example, an “effective amount” of a composition comprising gambogic acid may refer to an amount of the composition that has detectable cytotoxic activity against a tumor or cancer cell, that kills such cells, or displays another desired effect, e.g., to treat or prevent a cancer or another disease or disorder, or to treat the symptoms of a cancer or another disease or disorder, in a host.

The compositions and methods of the invention include such combinations of gambogic acid or an analogue or derivative thereof and one or more additional active ingredients, including but not limited to, various cancer therapeutics. For example, a single composition may include gambogic acid or an analogue or derivative thereof and one or more additional active ingredients. Alternatively, combination therapies may involve treatment with a composition comprising gambogic acid or an analogue or derivative thereof and another composition comprising one or additional active ingredients.

A combination of gambogic acid and another substance, e.g., another active ingredient, in a given composition or treatment may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the compositions and methods of the present invention. Such organisms include, but are not limited to, “mammals,” including, but not limited to, humans, monkeys, dogs, cats, horses, rats, mice, etc. In the context of the invention, the term “subject” generally refers to an individual who will receive or who has received treatment (e.g., administration of a compound of the invention, and optionally one or more anticancer agents) for cancer.

As used herein, “pharmaceutically acceptable salts” refer to derivatives of gambogic acid wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

Pharmaceutically acceptable salts of gambogic acid or other compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride as in Thomas J. Tucker, et al., J. Med. Chem. 1994 37, 2437-2444. A chiral compound of Formula I may also be directly synthesized using a chiral catalyst or a chiral ligand, e.g. Mark A. Huffman, et al., J. Org. Chem. 1995, 60, 1590-1594.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

As used herein, the terms “tumor” or “cancer” refer to a cellular disorder characterized by uncontrolled or disregulated cell proliferation, decreased cellular differentiation, inappropriate ability to invade surrounding tissue, and/or ability to establish new growth at ectopic sites. The terms “tumor” or “cancer” includes, but is not limited to, solid tumors and blood-borne tumors. The terms “tumor” or “cancer” encompass diseases of skin, tissues, organs, bone, cartilage, blood, and vessels. The terms “tumor” or “cancer” further encompass primary and metastatic cancers. Such cancers include, but are not limited to, cancers that express a protein that is a member of the Bcl-2 protein family.

Non-limiting examples of solid tumors that can be treated by the compositions and methods of the invention include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

In some other embodiments, the cancer is a hematologic malignancy. Non-limiting examples of hematologic malignancy include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

Besides cancers, gambogic acid may be used to treat non-cancerous proliferative lesions including, but not limited to, skin diseases, keloids, scars and benign prostatic hypertrophy.

Bcl-2 family proteins are known to cause autoimmunity in mice. Gambogic acid may be used to kill autoreactive lymphocytes in patients with autoimmune diseases and conditions. Therefore, in addition to cancer therapy, compositions comprising gambogic acid are useful for treating killing patients with autoimmune diseases and conditions, including, but not limited to, rheumatoid arthritis, lupus, multiple sclerosis, Sjögren's syndrome, spondylosing ankylitis, psoriasis, graft rejection, graft versus host disease, etc.

Obviously, numerous modifications and variations of the present invention are possible in light of the above teachings. It is therefore to be understood that within the scope of the appended claims, the invention may be practiced otherwise than as specifically described herein.

The Examples describe the use of various assays for determining whether a test compound competes with gambogic acid for binding to a Bcl-2-family protein, such as, for example, a fluorescence polarization assay and a time-resolved fluorescence resonance energy transfer assay. Other well-known assay methods may be used, including but not limited to an AlphaScreen™ (Ullman et al., Proc. Nat. Acad. USA 91:5426-5430, 1994; Ullman et al., Clin. Chem. 42:1518-1526, 1996; Bosse et al., Drug Discov. Today HTS Supplement 1:42-47, 2000) or a scintillation proximity assay (see, e.g., U.S. Pat. No. 4,382,074).

The compositions of the invention can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Such compositions may be systemically administered in vivo by a variety of routes. For example, they may be administered orally, in combination with a pharmaceutically acceptable excipients such as an inert diluent or an assimilable edible carrier. They may be enclosed in hard or soft shell gelatin capsules, may be compressed into tablets, or may be incorporated directly with the food of the patient's diet. For oral administration, the active ingredient or ingredients may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active ingredient in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compositions may also be administered intravenously or intraperitoneally by infusion or injection. Solutions of the cyclosporin, its salts and other active ingredients can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating gambogic acid or other active ingredients in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, cyclosporin A and other active ingredients may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of gambogic acid or other active ingredients can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of gambogic acid or other active ingredients of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other anticancer compounds will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 1 to about 75 mg/kg of body weight per day, or 1.5 to about 50 mg per kilogram body weight of the recipient per day, or about 2 to about 30 mg/kg/day, or about 2.5 to about 15 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

A patient may also be treated by ex vivo administration of compositions according to the present invention according to known protocols. Cells of the patient are removed from the patient, treated under suitable conditions with gambogic acid and, optionally, other agents as desired to kill the cancer cells, and returned to the patient's body.

The present invention will be further described by the following nonlimiting examples.

### Example 1

The natural product Gambogic acid (GA) has been reported to have cytotoxic activity against tumor cells in culture, and was identified as an active compound in a cell-based high-throughput screening (HTS) assay for activators of caspases, proteases involved in apoptosis. Using the anti-apoptotic Bcl-2-family protein, Bfl-1, as a target for screening of a library of natural products, we identified GA as a competitive inhibitor that displaced BH3 peptides from Bfl-1 in a fluorescent polarization assay (FPA). Analysis of competition for BH3 peptide binding revealed that GA inhibits all six human Bcl-2-family proteins to various extents, with Mcl-1 and Bcl-B the most potently inhibited (concentrations required for 50% inhibition [IC₅₀]<1 μM). Competition for BH3 peptide binding was also confirmed using a time-resolved fluorescence resonance energy transfer (TR-FRET) assay. GA functionally inhibited the anti-apoptotic Bcl-2-family proteins, as demonstrated by experiments using isolated mitochondria in which recombinant purified Bcl-2-family proteins suppress SMAC release in vitro, showing that GA neutralizes their suppressive effects on mitochondria in a concentration-dependent manner. GA killed tumor cell lines via an apoptotic mechanism, whereas analogs of GA with greatly reduced potency at BH3 peptide displacement showed little or no cytotoxic activity. However, GA retained cytotoxic activity against bax−/− bak−/− cells in which anti-apoptotic Bcl-2-family proteins lack a cytoprotective phenotype, implying that GA also has additional targets that contribute to its cytotoxic mechanism. Altogether, the findings suggest that suppression of anti-apoptotic Bcl-2-family proteins may be among the cytotoxic mechanisms by which GA kills tumor cells.

### Materials and Methods

Compounds. The MicroSource Spectrum Collection Library (Discovery Systems, Inc., Gaylordsville, CT) is a ˜2000 compound collection of mostly pure natural products and their derivatives. Compounds were supplied as 10 mM stocks of Me2SO, stored at −20° C. and thawed immediately before analysis. Aliquots were dissolved to a final concentration of 10 μM for fluorescence polarization assays. Gambogic acid was purchased from Calbiochem. All the gambogic acid analogs were purchased from MicroSource.

Protein Purification. GST-fusion proteins containing Bcl-X_{L}, Bcl-2, Bcl-W, Bcl-B, Bfl-1 and Mcl-1 lacking their C-terminal transmembrane domains (last 20 amino-acids) (“ΔTM”) were expressed from pGEX 4T-1 plasmid in XL-1 Blue cells (Stratagene, Inc.) as described previously (Zhai et al., Cell Death Differ. 13:1419-1421, 2006). Briefly, cells were grown in 2 L of LB with 50 μg/mL ampicillin at 37° C. to an OD600 nm of 1.0, then IPTG (0.5 mM) was added, and the cultures were incubated at 25° C. for 6 h. Cells were then recovered in 20 mM phosphate buffer (pH 7.4), 150 mM NaCl, 1 mM DTT, 1 mM EDTA, 1 mM PMSF, followed by sonication. Cellular debris was sedimented by centrifugation at 27,500 g for 20 min, and the resulting supernatants were incubated with 10 mL of glutathionine-Sepharose (Pharmacia) at 4° C. for 2 h. The resin was washed 3 times with 20 mM phosphate buffer (pH 7.4), 150 mM NaCl, and 1 mM DTT, and then 10 mM of reduced glutathione dissolved in 50 mM Tris-HCl (pH 8.0) was used to elute the GST-fusion proteins.

Other recombinant proteins used here, including His₆-Bid and His₆-caspase 8, were expressed and purified using methods similar to prior publications (Zhai et al., J. Biol. Chem. 280:15815-15824, 2005).

Fluorescence Polarization Assays (FPAs). FPAs were performed as described previously using various Bcl-2-family proteins and fluorescein isothiocyanate (FITC)-conjugated Bid BH3 peptide (Zhai et al., Cell Death Differ. 13:1419-1421, 2006; Zhai et al., Biochem. J. 376 (Pt. 1):229-236, 2003). Briefly, Bcl-2 proteins were incubated with 5 nM of FITC-Ahx-EDIIRNIARHLAQVGDSMDR in the dark. Fluorescence polarization was measured using an Analyst TM AD Assay Detection System (LJL Biosystem, Sunnyvale, Calif.) in phosphate-buffered saline (PBS) [pH 7.4]. IC₅₀ determinations were performed using GraphPad Prism software (GraphPad, Inc., San Diego, Calif.).

Competitive Peptide Displacement Assays. Methods for competitive peptide displacement assays were similar to previous publications (Zhai et al., Cell Death Differ. 13:1419-1421, 2006). Briefly, 100 nM of GST-Bcl-2 proteins were incubated with the compounds at various concentrations for 5 min at room temperature in PBS. Then 5 nM of FITC-Bid BH3 peptide was added and fluorescence polarization was measured after 10 min. IC₅₀ determinations were generated by fitting the experimental data using a sigmoidal dose-response nonlinear regression model with GraphPad Prism software (GraphPad, Inc., San Diego, Calif.).

Time-Resolved-Fluorescence Resonance Energy Transfer (TR-FRET) Assays. For TR-FRET assays, GST-Bcl-X_{L} and anti-GST-terbium (Invitrogen) were mixed together with the FITC-Bad BH3 peptide in PBS containing 0.005% tween 20 in 96 well plates in a total volume of 20 μl per well. After incubation at room temperature for 30 min, 2 μl of gambogic acid-containing solutions were added to the reaction mixtures containing 10 nM of Bcl-X_{L}, 10 nM of FITC-Bad BH3 peptide and 2 nM of anti-GST-terbium for 30 min at room temperature. TR-FRET signals were measured with a SpectraMax M5 plate reader (Molecular Devices) using the following settings: excitation at 330 nm, emission for FITC signal at 490 nm, and emission for terbium signal at 520 nm.

Mitochondria Purification and Protein Release Assays. HeLa cells were pelleted by centrifugation, and then washed once in HM buffer (10 mM HEPES, pH 7.4, 250 mM mannitol, 10 mM KCl, 5 mM MgCl₂, 1 mM EGTA), containing 1 mM PMSF and a mixture of protease inhibitors (Roche Molecular Biochemicals). The cell pellet was then homogenized in HM buffer by 50 strokes of a dounce homogenizer, using a B-type pestle. The homogenate was centrifuged twice at 600 g for 5 min to remove nuclei and debris. The resulting supernatant was centrifuged at 10,000 g for 10 min, and the resulting mitochondria-containing pellet was washed twice with the HM buffer.

For mitochondrial protein release assays, 10 μl of mitochondria (50 μg) were added into a final volume of 50 μl HM buffer containing gambogic acid, tBid or tBid pre-incubated with gambogic acid or Bcl-2 family proteins at 30° C. for 15 min. The reactions were further incubated at 30° C. for 40-60 min, then mitochondria were pelleted by centrifugation and the supernatants were collected, boiled in Laemmli sample buffer, and analyzed by SDS-PAGE/immunoblotting using anti-SMAC antibody (Zhai et al., J. Biol. Chem. 280:15815-15824, 2005).

Peptide Synthesis. Peptides were synthesized using Fmoc solid-phase synthesis on an ACT 350 multiple peptide synthesizer. FITC conjugated-Bid were synthesized on Fmoc-Alanine Wang resin to give peptides with C-terminal carboxyl groups. FITC was linked to the Ahx. The crude peptides were purified with a Gilson HPLC instrument and analyzed by MALDI-TOF mass analysis with an Applied Biosystems Voyager System 6264.

Cell Culture, Transfection, and Apoptosis Assays. HeLa (cervical cancer), HL60 (acute myeloid leukemia), Jurkat (T cell leukemia), PPC1 (prostate cancer) and MEF (mouse embryonic fibroblast) cells were maintained in Dulbecco's modified Eagle's medium (Irvine Scientific) supplemented with 10% fetal bovine serum (FBS), 1 mM L-glutamine, and antibiotics. Apoptosis was assessed using staining with Annexin V-FITC and propidium iodide (PI), followed by flow-cytometry analysis using FL-1 and FL-3 channels of a flow cytometer (Becton Dickinson; FACSort; San Jose, Calif.). Annexin V-positive/PT-negative cells were considered apoptotic.

For caspase assays, cell lysates were prepared, normalized for protein content, and 10 μg aliquots of cell lysates were incubated with 100 μM DEVD-AFC, measuring enzyme activity by the release of AFC-fluorescence. Data are reported as relative fluorescence units (RFU) of product produced per min per μg of total protein.

### Results and Discussion

Identification of GA as a Bfl-1-inhibitory compound by HTS. We devised a HTS in which binding of a FITC-conjugated BH3 peptide to recombinant purified Bfl-1 protein is measured by FPA. A library of 2000 natural products was screened for suppression of BH3 peptide binding by ≧50% using this FPA, resulting in ˜30 hits, among which was gambogic acid (GA).

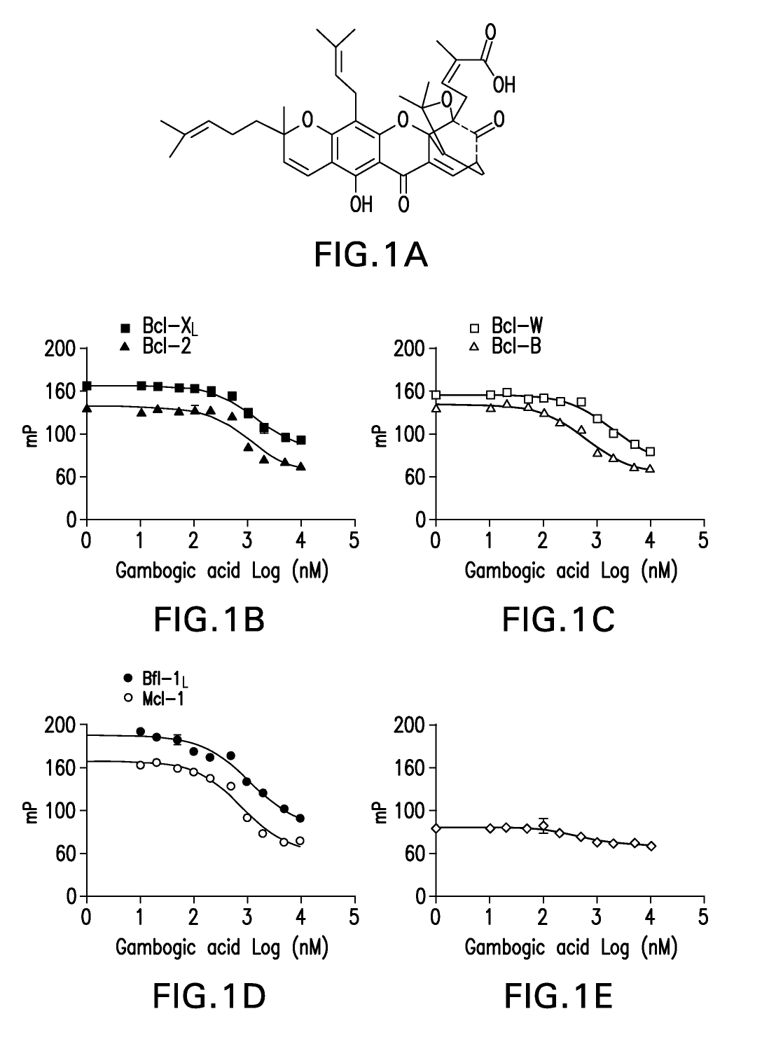

GA competes for BH3 peptide binding to the six anti-apoptotic Bcl-2-family proteins with variable efficiencies. The activity of GA against the six human anti-apoptotic Bcl-2-family proteins was contrasted, using FPAs we previously established (Zhai et al., Cell Death Differ. 13:1419-1421, 2006). GA displaced to various extents FITC-BH3 peptide binding to all six proteins, with Mcl-1 and Bcl-B showing the greatest sensitivity (apparent IC₅₀<1 μM) and Bcl-2 the least (FIG. 1). Due to solubility, we were unable to increase the GA concentration to achieve complete BH3 peptide displacement for some Bcl-2 family proteins in these assays. Incubating GA with FITC-BH3 peptide in the absence of Bcl-2-family proteins only marginally affected baseline (background) fluorescence polarization (˜Δ20 mP at concentrations≧20 μM), excluding a direct effect of GA on the peptide probe as an explanation for the results (FIG. 1). In contrast to Bcl-2-family proteins, GA did not inhibit in FPAs using rhodamine-SMAC peptide binding to BIR3 of XIAP (not shown) or FITC-ATP binding to Hsp70 (not shown), thus demonstrating the specificity for anti-apoptotic Bcl-2-family proteins.

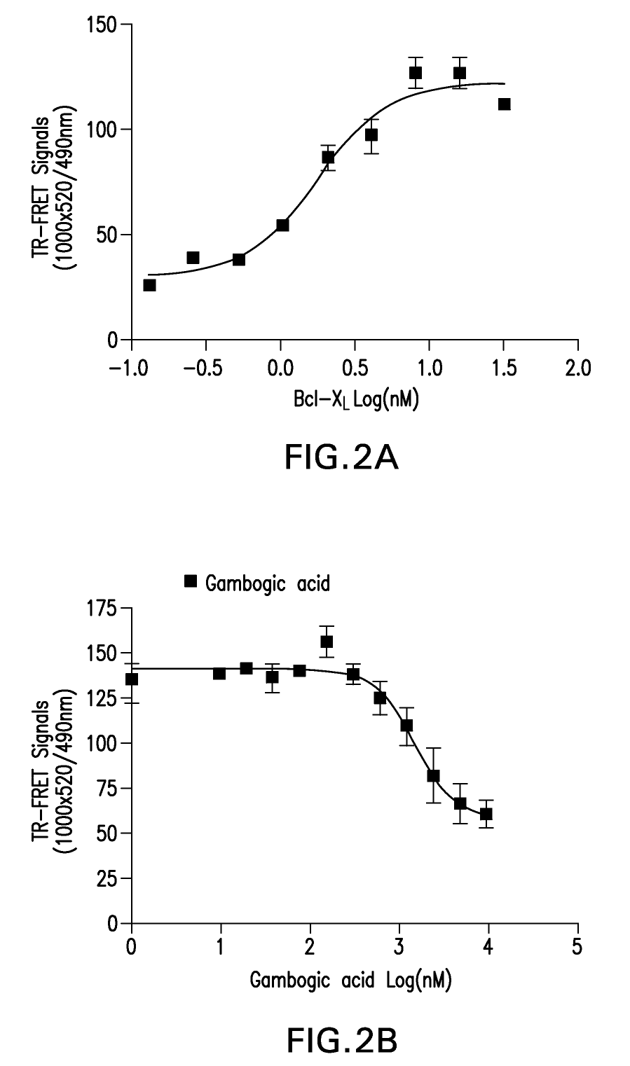

TR-FRET confirmation of GA competition for BH3-binding site. To confirm by an independent method the ability of GA to compete with BH3 peptides for binding to Bcl-2-family proteins, we devised TR-FRET assays for Bcl-X_{L}, in which FITC-Bad BH3 peptide bound to recombinant purified GST-Bcl-X_{L} is excited by emissions from terbium conjugated anti-GST antibody. FITC-Bad BH3 peptide bound to GST-Bcl-X_{L} (but not GST [not shown]) in a concentration and saturable manner, with apparent Kd's of 2 nM (FIG. 1A). GA inhibited FITC-Bad BH3 peptide binding to GST-Bcl-X_{L}, as measured by TR-FRET, with apparent IC₅₀'s of 1.5 μM (FIG. 2B), thus yielding similar results as those obtained using FPAs.

GA neutralizes activity of Bcl-2-family proteins in vitro. An active, N-terminally truncated form of the pro-apoptotic BH3-containing protein Bid (tBid) induces release of apoptogenic proteins such as SMAC from isolated mitochondria in vitro (Becattini et al., Chem. Biol. 11:1107-1117, 2004; Zhai et al., J. Biol. Chem. 280:15815-15824, 2005; Luciano et al., J. Biol. Chem. 280:15825-15835, 2005). Using this assay, we showed that five of the six human anti-apoptotic Bcl-2 family proteins negate tBid-induced release of SMAC from isolated mitochondria. tBid (20 ng, cleaved by caspase 8) was pre-incubated with 2 μg of Bcl-X_{L}, Bcl-2, Bcl-W, Bcl-B, Bfl-1 or Mcl-1 together with various concentrations of GA for 15 min in HM buffer, before adding 50 μg of HeLa mitochondria for 1 hr at 30° C. Samples were centrifuged to generate supernatants that were analyzed by SDS-PAGE/immunoblotting using anti-SMAC antibody. For these five proteins (Bcl-2, Bcl-X_{L}, Bfl-1, Bcl-W, Mcl-1), adding GA restored tBid-induced SMAC release in a concentration-dependent manner. Complete restoration was typically achieved with 5 μM GA, representing an approximately 10:1 molar excess of GA relative to anti-apoptotic Bcl-2-family proteins. GA also enhanced tBid-induced release of SMAC from isolated mitochondria when recombinant Bcl-2-family proteins were not added, suggesting it may neutralize endogenous anti-apoptotic Bcl-2-family proteins associated with mitochondria.

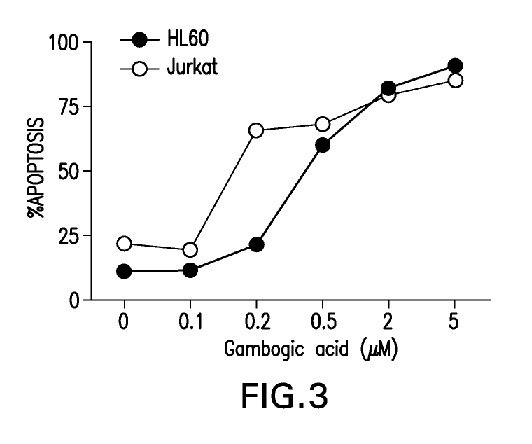

GA overcomes cytoprotection of Bcl-2 in leukemia cells. GA has cytotoxic activity against various tumor cell lines in culture and induces apoptosis (Zhang et al., Bioorg. Med. Chem. 12:309-317, 2004; Zhao et al., Biol. Pharm. Bull. 27:998-1003, 2004). We confirmed the apoptosis-inducing activity of GA using Jurkat T-cell acute lymphoblastic leukemia and HL-60 acute promyelomonocytic leukemia cell lines, using Annexin V/propidium iodide (PI) staining, counting Annexin V+/PT− cells as apoptotic (FIG. 3). The concentration of GA required to induce apoptosis of 50% of the cells within 20-24 hrs was ˜0.2 μM and ˜0.5 μM for Jurkat and HL-60, respectively.

To determine GA's affect on processing of pro-caspase-3, a marker of apoptosis, cells of the prostate cancer cell line PPC1 were treated with gambogic acid at concentrations from 0 to 10 μM. After 20 hrs, the cells were collected and the cell lysates were analyzed by SDS-PAGE/immunoblotting using anti-caspase 3 antibody. GA induced clearly detectable proteolytic processing of pro-caspase-3 at concentrations≧2 μM.

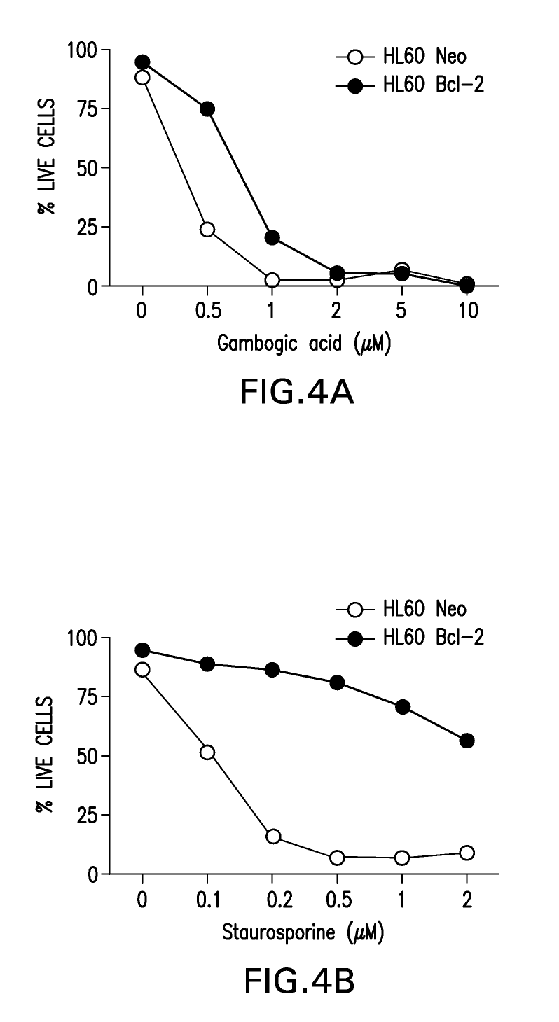

We tested the cytotoxic activity of GA against HL-60 cells that had been stably transfected with NEO-control or Bcl-2-expression plasmids (Konopleva et al., Blood 95:3929-3938, 2000). Over-expression of Bcl-2 reduced the sensitivity of HL-60 cells to GA, shifting the dose-response curve to the right, such that higher concentrations of GA were required to kill the cells (FIG. 4A). Treating HL-60 NEO-control and Bcl-2-transfected cells with an upstream activator of the mitochondrial pathway for apoptosis, staurosporine (STS), confirmed that Bcl-2 over-expressing HL-60 cells have a block to apoptosis (FIG. 4B), which GA overcomes.

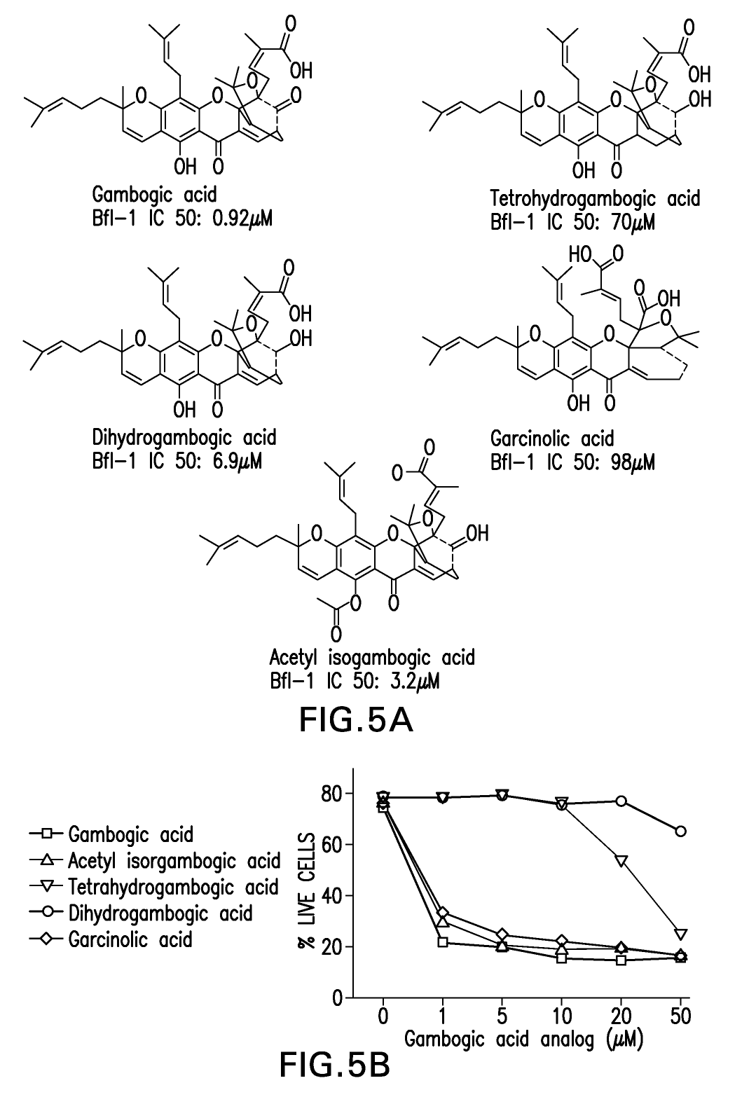

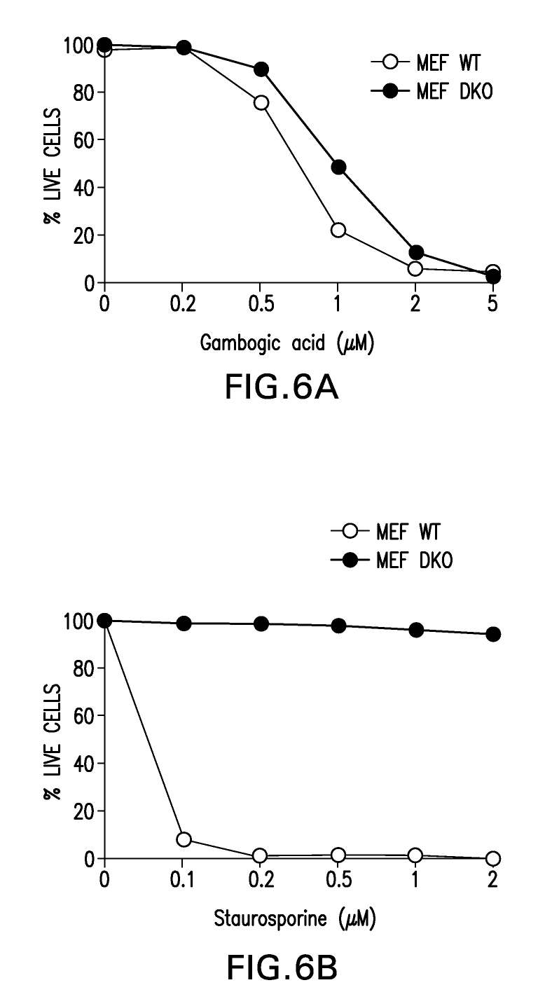

GA analogs show differential activity against Bcl-2-family proteins correlating with cytotoxicity. We examined the activity of analogs of GA with respect to displacement of Bid BH3 peptide from Bfl-1 in vitro (FIG. 5A) and cytotoxic activity (FIG. 5B). In the FPAs, dihydro-GA and acetyl-iso-GA displaced BH3 peptide from Bfl-1 with potencies only 3-6 fold less that GA (FIG. 6A). In contrast, tetrahydro-GA and Garcinolic acid had greatly reduced activity in BH3 displacement assays. In agreement with the BH3 displacement data, dihydro-GA and acetyl-iso-GA exhibited cytotoxic activity against cultured leukemia cells, while tetrahydro-GA showed markedly reduced activity (˜50-fold less potent) and Garcinolic acid was non-toxic (FIG. 5B). Thus, the cytotoxic activity of GA analogs correlates roughly with their ability to compete with BH3 peptides for binding to an anti-apoptotic Bcl-2-family member.

GA-induced cytotoxicity is only partly Bcl-2-dependent. Anti-apoptotic Bcl-2-family proteins suppress mitochondria-initiated cell death by inhibiting pro-apoptotic Bcl-2 members Bax and Bak (Kroemer and Reed, Nat. Med. 6:513-519, 2000; Reed, Am. J. Pathol. 157:1415-1430, 2000; Li et al., Cell 116 (2 Suppl.):S57-59, 2 p. following S59). Consequently, when cells are genetically engineered to lack Bax and Bak, then Bcl-2 and related cytoprotective proteins no long display an anti-apoptotic phenotype (Wei et al., Science 292:727-730, 2001). Cells doubly deficient in Bax and Bak therefore provide a context for assessing the mechanism of GA-induced toxicity. GA killed transformed mouse fibroblasts (MEFs) generated from bax+/+ bak+/+ embryos in a concentration-dependent manner (FIG. 6A). However, GA also killed bax−/− bak−/− MEFs, requiring only approximately two-fold higher concentrations of compound to reach the Lethal Dose 50% (LD₅₀). Treating these MEFs with STS confirmed that Bak/Bak double knock-out cells have a profound block to apoptosis (FIG. 6B), which GA overcomes. We conclude therefore that GA induces cytotoxicity through mechanisms that are only partly dependent on Bcl-2-family proteins.

IC₅₀ of GA for six anti-apoptotic human Bcl-2 family proteins. Table 1 provides a summary of the IC₅₀ of gambogic acid assessed using FPAs for all six anti-apoptotic human Bcl-2 family proteins. IC₅₀ determinations were generated by fitting experimental data from FPAs, using a sigmoidal dose-response nonlinear regression model.

TABLE 1
Summary of IC₅₀ of gambogic acid assessed using FPAs
for six anti-apoptotic human Bcl-2 family proteins.

| Anti-apoptotic | IC₅₀ (μM) |
|---|---|
| Bcl-2 Protein | for Gambogic acid |
| Bcl-X_{L} | 1.47 |
| Bcl-2 | 1.21 |
| Bcl-W | 2.02 |
| Bcl-B | 0.66 |
| Bfl-1 | 1.06 |
| Mcl-1 | 0.79 |

### Summary

We have demonstrated that the cytotoxic natural product GA competes for BH3 peptide binding sites on several anti-apoptotic members of the Bcl-2 family and neutralizes the ability of these proteins to suppress release of apoptogenic proteins from isolated mitochondria. Structure-function analysis (SAR) of GA using analogs suggested a general correlation between BH3 competition and cytoxicity activity, but experiments with bax/bak double knock-out cells suggest GA-induced cytotoxicity is only partially dependent on Bcl-2-family proteins.

GA has been reported to affect other molecular events relevant to cytotoxicity, including inducing increases in Bax protein levels, reductions in Bcl-2 protein levels, and suppressing transferring receptor internalization (Zhao et al., Biol. Pharm. Bull. 27:998-1003, 2004; Kasibhatla et al., Proc. Natl. Acad. Sci. USA 102:12095-12100, 2005). GA also reportedly induces G2/M-phase arrest of dividing cells (Ye et al., Carcinogenesis 28:632-638, 2007), suggesting that this natural product hits targets involved in cell cycle. Thus, like many natural products, GA may have several targets in mammals, among which are anti-apoptotic members of the Bcl-2 family.

It is interesting to speculate why plants might produce compounds that neutralize Bcl-2 family proteins. Insects, nematodes, and other animal species that eat plants contain evolutionarily conserved anti-apoptotic Bcl-2 family proteins (reviewed in Reed et al., Sci. STKE 2004 (239):re9, 2004). Consequently, plants might produce antagonists of these compounds as a mechanism to defend themselves against animal species. In this regard, several natural products from diverse plant species have been shown to competitively displace BH3 peptides and neutralize the activity of anti-apoptotic Bcl-2-family proteins, including epigallocatechin gallate (EGCG) from green tea, theaflavins from black tea, and gossypol from cotton seeds (Leone et al., Cancer Res. 63:8118-8121, 2003; Kitada et al., J. Med. Chem. 46:4259-4264, 2003). These compounds are structurally diverse, but all display <1 μM activity against a variety of human anti-apoptotic Bcl-2 family proteins. It remains to be determined whether their affinity is greater for Bcl-2 family members from insects or other lower-organisms of the animal kingdom.

### REFERENCES

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method for identifying a test compound that is effective for treating a disease or condition of a mammal selected from the group consisting of a cancer, a non-cancerous proliferative condition, and an autoimmune disorder, the method comprising determining whether the test compound competes with gambogic acid or a gambogic acid derivative for binding to a Bcl-2-family protein.

2. The method of claim 1 comprising:
(a) providing a first mixture comprising the Bcl-2-family protein and the test compound;
(b) incubating the first mixture under conditions suitable for binding of the test compound to the Bcl-2-family protein;
(c) adding the gambogic acid or the gambogic acid derivative to the mixture to produce a second mixture comprising the Bcl-2-family protein, the gambogic acid or the gambogic acid derivative, and the test compound;
(d) incubating the second mixture under conditions suitable for binding of the gambogic acid or the gambogic acid derivative to the Bcl-2-family protein; and
(e) measuring binding of the gambogic acid or the gambogic acid derivative to the Bcl-2-family protein in the second mixture.

3. The method of claim 1 comprising:
(a) providing a first mixture comprising the Bcl-2-family protein and the gambogic acid or the gambogic acid derivative;
(b) incubating the first mixture under conditions suitable for binding of the gambogic acid or the gambogic acid derivative to the Bcl-2-family protein;
(c) adding the test compound to the first mixture to produce a second mixture comprising the Bcl-2-family protein, the gambogic acid or the gambogic acid derivative, and the test compound;
(d) incubating the second mixture under conditions suitable for binding of the test compound to the Bcl-2-family protein; and
(e) measuring binding of the gambogic acid or the gambogic acid derivative to the Bcl-2-family protein.

4. The method of claim 1 wherein the gambogic acid or the gambogic acid derivative comprises a detectable label.

5. The method of claim 4 wherein the label is selected from the group consisting of a fluorochrome, biotin and a radiolabel.

6. The method of claim 5 wherein the label is a fluorochrome.

7. The method of claim 6 wherein determining whether the test compound competes with the gambogic acid or the gambogic acid derivative for binding to the Bcl-2-family protein comprises performing an assay to measure binding of the gambogic acid or the gambogic acid derivative to the Bcl-2-family protein, wherein the assay is selected from the group consisting of a fluorescence polarization assay, a time-resolved fluorescence resonance energy transfer assay, an AlphaScreen™, and a scintillation proximity assay.

8. The method of claim 1 further comprising contacting a cell of the mammal that expresses a Bcl-2 family protein with the test compound and determining whether the test compound is cytotoxic against the cell.

9. The method of claim 8 wherein the cell is selected from the group consisting of a HeLa cell, a HL60 cell, a Jurkat cell, and a PPC1 cell.

10. The method of claim 8 comprising providing the cell in a well of a multi-well plate.

11. The method of claim 1 wherein the cancer is selected from the group consisting of pancreatic cancer; bladder cancer; colorectal cancer; breast cancer, including metastatic breast cancer; prostate cancer; renal cancer; hepatocellular cancer; lung cancer; ovarian cancer; cervical cancer; gastric cancer; esophageal cancer; head and neck cancer; melanoma; neuroendocrine cancer; brain tumors; bone cancer; soft tissue sarcoma; acute myeloid leukemia; chronic myelogenous leukemia; acute lymphoblastic leukemia; chronic lymphocytic leukemia; Hodgkin's disease; non-Hodgkin's lymphoma; B-cell lymphoma; T-cell lymphoma; multiple myeloma; Waldenstrom's macroglobulinemia; myelodysplastic syndromes; and myeloproliferative syndromes.

12. The method of claim 1 wherein the non-cancerous proliferative condition is selected from the group consisting of a skin disease, a keloid, a scar and benign prostatic hypertrophy.

13. The method of claim 1 wherein the autoimmune disorder is selected from the group consisting of rheumatoid arthritis, lupus, multiple sclerosis, Sjögren's syndrome, spondylosing ankylitis, psoriasis, graft rejection and graft versus host disease.

14. The method of claim 1 wherein the mammal is a human.

15. An automated method of claim 1.

16. A method for identifying a compound that effective for treating a mammal having a disease or condition selected from the group consisting of a cancer, a non-cancerous proliferative condition, and an autoimmune disorder comprising comparing a three-dimensional structure of said compound when bound to a Bcl-2-family protein to a three-dimensional structure of gambogic acid bound to the Bcl-2-family protein.

17. A composition comprising an amount of gambogic acid or a gambogic acid derivative that is effective in treating a mammal having a disease or condition selected from the group consisting of a cancer, a non-cancerous proliferative condition, and an autoimmune disorder.

18. The composition of claim 17 wherein the cancer is selected from the group consisting of pancreatic cancer; bladder cancer; colorectal cancer; breast cancer, including metastatic breast cancer; prostate cancer; renal cancer; hepatocellular cancer; lung cancer; ovarian cancer; cervical cancer; gastric cancer; esophageal cancer; head and neck cancer; melanoma; neuroendocrine cancer; brain tumors; bone cancer; soft tissue sarcoma; acute myeloid leukemia; chronic myelogenous leukemia; acute lymphoblastic leukemia; chronic lymphocytic leukemia; Hodgkin's disease; non-Hodgkin's lymphoma; B-cell lymphoma; T-cell lymphoma; multiple myeloma; Waldenstrom's macroglobulinemia; myelodysplastic syndromes; and myeloproliferative syndromes.

19. The composition of claim 17 wherein the non-cancerous proliferative condition is selected from the group consisting of a skin disease, a keloid, a scar and benign prostatic hypertrophy.

20. The composition of claim 17 wherein the autoimmune disorder is selected from the group consisting of rheumatoid arthritis, lupus, multiple sclerosis, Sjögren's syndrome, spondylosing ankylitis, psoriasis, graft rejection, and graft versus host disease.

21. The composition of claim 17 wherein the mammal is a human.

22. The composition of claim 17 comprising a pharmaceutically acceptable carrier.

23. The composition of claim 17 comprising an amount of an additional active ingredient that is effective in treating the mammal having said disease or condition.

24. A method of treating a cancer in a mammal in need thereof comprising administering to the mammal a composition comprising an amount of gambogic or a gambogic acid derivative that is cytotoxic against the cancer.

25. The method of claim 24 wherein the cancer is selected from the group consisting of pancreatic cancer; bladder cancer; colorectal cancer; breast cancer, including metastatic breast cancer; prostate cancer; renal cancer; hepatocellular cancer; lung cancer; ovarian cancer; cervical cancer; gastric cancer; esophageal cancer; head and neck cancer; melanoma; neuroendocrine cancer; brain tumors; bone cancer; soft tissue sarcoma; acute myeloid leukemia; chronic myelogenous leukemia; acute lymphoblastic leukemia; chronic lymphocytic leukemia; Hodgkin's disease; non-Hodgkin's lymphoma; B-cell lymphoma; T-cell lymphoma; multiple myeloma; Waldenstrom's macroglobulinemia; myelodysplastic syndromes; and myeloproliferative syndromes.

26. A method of making a medicament useful in treating a cancer in a mammal in need of such treatment, the method comprising incorporating a compound that competes with gambogic acid or a gambogic acid derivative for binding to a Bcl-2-family protein in a cell of the cancer into a pharmaceutical composition comprising the compound and a pharmaceutically acceptable carrier.
